(19) United States
(12) Patent Application Publication
(10) Pub. No.: US 2010/0305413 A1
(43) Pub. Date: Dec. 2, 2010

(54) SENSING APPARATUS

(76) Inventor: William Paterson, Oxfordshire (GB)

Correspondence Address: FISH & RICHARDSON P.C., PO BOX 1022, MINNEAPOLIS, MN, 55440-1022, US

(21) Appl. No.: 12/672,586

(22) PCT Filed: Aug. 5, 2008
(86) PCT No.: PCT/GB08/02666

§ 371 Date: Apr. 23, 2010

(30) Foreign Application Priority Data
Aug. 9, 2007 (GB) 0715590.6

(51) Int. Cl. A61B 5/145; B29C 35/08

(52) U.S. Cl. 600/301; 264/400; 600/365

(57) ABSTRACT

A process for preparing a tube for insertion into the human or animal body, said process comprising: (a) providing a hollow tube comprising a first material and a second material, the first material being in the form of a coil or a tubular mesh, and the second material coating the first material in order to form a continuous substantially impermeable outer wall of the hollow tube; (b) selectively removing a portion of said second material in order to generate at least one opening in a region of the outer wall, while retaining the first material in said region.

The present invention relates to porous tubing for use in sensing apparatus, and includes a sensor for detecting an analyte which utilises such tubing. Also described is a method for preparing the porous tubing. The invention relates in particular to invasive or implantable sensors.

### BACKGROUND TO THE INVENTION

A multitude of chemical and biosensors are known, in particular in the field of invasive and implantable sensors. These can be inserted into the body, for example into tissue, a body cavity, a blood vessel or another bodily fluid, and used to monitor one or more variables such as temperature and/or an analyte concentration. Measurement of analytes such as saccharides e.g. glucose, oxygen, carbon dioxide and pH can be of great importance during surgery, post-operatively and during hospitalisation under intensive care.

Prior art sensors include probes comprising a tube in which the sensing apparatus is located, and which can be inserted into tissue, into a blood vessel or into a body cavity. The tube containing the sensing apparatus has a continuous wall, without any breaks or holes within its structure, hence sealing the sensing apparatus inside. Where the sensor is intended to measure the presence of an analyte, the analyte can enter the tube at its open end, travelling along the tube to the sensing apparatus. A suitable configuration for this type of prior art sensor is shown in FIG. 1. FIG. 1 depicts the sensing tip of the sensor only, including a sensor and a thermistor.

These prior art sensors are not without problems, and in particular the probes can be exposed to unacceptable physical stresses, resulting in kinking, compression and breakage. There has therefore been a need to provide stronger and more robust sensors which can withstand the pressures of being introduced into the body, yet which retain some flexibility allowing them to be accurately positioned in the correct region for sensing. This problem has been addressed through the provision of sensors having reinforced walls, which are better able to withstand the stresses to which they are exposed. The reinforced walls can be provided in a number of ways, for example by providing a braided tubular structure which contains the sensing apparatus, as described in International patent publication no. WO-A-2004/054438. The braid is able to provide strength to the sensor, while allowing the tubular structure to flex and be manoeuvred to the correct sensing position.

While such braided sensors have shown benefits over previous sensors, they are not without disadvantages. In particular, the structured surface which results from the use of a braided tubular structure increases incompatibility of the sensor with the tissue or vessel into which it is introduced. For example, a braided surface can lead to fibrinogen formation and possible clot formation, and can also disrupt blood flow through a blood vessel where the sensor is located. Furthermore, a braided surface can be abrasive, and can tear or snag a patient's skin or tissue during introduction of the sensor into, and movement within, the body.

There is therefore a need to provide new sensing apparatus which is robust and can be introduced and positioned accurately within the body, and which is compatible with the environment into which it is introduced.

### SUMMARY OF THE INVENTION

The present invention provides a process for preparing a tube for insertion into the human or animal body, said method comprising:
(a) providing a hollow tube comprising a first material and a second material, the first material being in the form of a coil or a tubular mesh, and the second material coating the first material in order to form a continuous substantially impermeable outer wall of the hollow tube;
(b) selectively removing a portion of said second material in order to generate at least one opening in a region of the outer wall, while retaining the first material in said region.

The invention also provides a tube for insertion into the human or animal body which is preparable according to the process described above. There is further provided tube for insertion into the human or animal body, said tube comprising a first material in the form of a coil or a tubular mesh, and a second material coating the first material in order to form a continuous substantially impermeable outer wall of the hollow tube, wherein at least a portion of the second material has been selectively removed in order to generate at least one opening in a region of the outer wall, while retaining the first material in said region.

The invention also provides a sensor device comprising a tube as described above, wherein said tube houses a probe comprising at least one analyte sensor.

The tubes provided above are advantageous because they have the robustness and manoeuvrability necessary for insertion and use within the body, yet have a substantially continuous outer wall. Accordingly, sensors which employ these tubes are able to be easily introduced into the body yet are compatible with the region into which they are introduced, reducing the possibility of fibrinogen or blood clot formation and reducing the likelihood of tearing of the patient's skin or tissue.

### BRIEF DESCRIPTION OF THE FIGURES

FIG. 1 depicts a sensor according to the prior art.

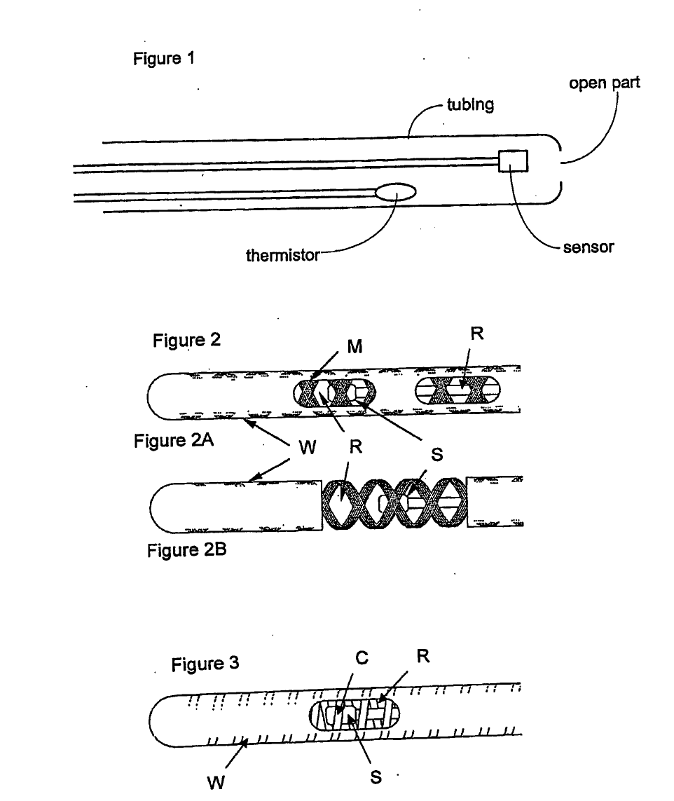

FIGS. 2 to 4 depict sensors according to various embodiments of the present invention.

### DETAILED DESCRIPTION OF THE INVENTION

The first material is in the form of a coil or a tubular mesh. Suitable materials for use as the first material include both metallic and non-metallic materials. Suitable metallic materials include stainless steel, gold, titanium and silver, and alloys such as nitinol, beryllium copper and MP-35-N alloys comprising cobalt, nickel, chromium, and molybdenum. Stainless steel is preferred. Suitable non-metallic materials include synthetic polymers such as polyamides, polyesters, polyurethanes, polyolefins, nylon and fluoropolymers, for example polytetrafluoroethylene (PTFE). The first material must, however, be chosen so that it is possible to selectively remove a region of the second material which coats the first material, without the first material itself being removed in that region.

As mentioned above, the first material can be in the form of a coil or a tubular mesh. When in the form of a coil, the coil can be made from wires of the first material which preferably have either round or flat cross-section. When in the form of a tubular mesh, the mesh structure advantageously comprises a number of filaments. The term “filaments” is used to refer to any elongate strand irrespective of its cross-sectional configuration and structure. For example, the filaments may be round or flat in cross-section. In one embodiment, the mesh can comprise a number of helically wound filaments, for example comprising a first group of filaments wound in an anticlockwise direction and a second group of filaments wound in an opposite, clockwise direction. Suitable mesh structures are described in International publication no. WO-A-2004/054438.

Where the first material is in the form of a mesh, the density of filament crossovers may be varied in order to control the properties of the resulting tube. For example, a high density mesh may have greater strength and a low density mesh a greater flexibility. Variation in mesh density will also vary the porosity of the mesh. This is significant at the location of the opening in the outer wall since the porosity of the mesh will control the speed of diffusion of the material to be tested into the tube. Variation in the tightness of a coil can provide a similar effect.

The second material is used to coat the first material in order to form a continuous substantially impermeable outer wall of the hollow tube. As used herein, the phrase substantially impermeable means that the second material forms an effectively closed tube, which is impermeable to the ingress of material from outside the tube to inside the tube. Accordingly, until a portion of the second material is removed, the tube is effectively sealed along its length, except at its ends.

Suitable materials for use as the second material generally include polymeric materials, more particularly polyesters, polyolefins such as polyethylene (PE), e.g. low density polyethylene (LDPE), fluoropolymers such as fluorinated ethylene propylene (FEP), polytetrafluoroethylene (PTFE) and perfluoroalkoxy polymer (PFA), polyvinylchloride (PVC), polyamides such as polyether block amide (PEBA), Pebax®, nylon and polyurethane. Polyesters and polyolefins are preferred due to their suitability for extrusion over the coil or tubular mesh. The selective removal of a portion of a polyester or polyolefin coating, e.g. by laser ablation, is also straightforward. Polyolefins are particularly preferred due to the ease of laser ablating these materials.

In order to form a continuous substantially impermeable tube prior to selective removal of a portion of the second material, the second material is first used to coat the coil or tubular mesh formed by the first material. The second material can either coat the outer surfaces of the first material, and in effect form a continuous substantially impermeable tube around the coil or tubular mesh formed by the first material, or the second material can entirely encapsulate the first material, effectively forming a tube of the second material in which is embedded the coil or tubular mesh formed by the first material. In one embodiment the second material can be applied to the first material by dip coating the coil or tubular mesh formed by the first material. In this embodiment, the second material is probably a polyimide, which results in a very stiff tube. In another embodiment a tube of the second material can be provided, around which is formed the coil or tubular mesh of the first material. A further layer of the second material is then applied over the first material, resulting in the first material being sandwiched between two layers of the second material.

As mentioned above, it is necessary that it be possible to selectively remove a region of the second material while retaining the first material in that region. Accordingly, it is a requirement that the second material is different from the first material. By “different from” we mean that the first and second materials must have a difference in properties such that it is possible to selectively remove a region of the second material. This difference may be achieved by using an entirely different material, or by using the same material but using different forms which have different physical properties. Preferably entirely different materials are used. For example, in a preferred embodiment the first material is metallic and the second material is polymeric.

In addition to the first and second materials, it is possible to include further materials in the tubes of the invention. For example, for some applications it may be useful to include a radio opaque additive to enable the sensor incorporating the tube to be visible in vivo. For example, radio opaque additives such as barium sulfate, bismuth subcarbonate, bismuth trioxide and tungsten can be added. Where present, these are preferably doped within the second material.

In the processes of the invention, a portion of the second material is selectively removed in order to generate at least one opening in a region of the outer wall, while retaining the first material in that region. As the first material is present in the form of a coil or a tubular mesh, the first material does not form a completely closed tube. Accordingly, when the second material is removed in said region, this effectively forms a break in the continuous substantially impermeable wall of the tube. Where the second material simply coats the first material, it is necessary simply to remove the coating provided by this second material in the region where the opening is to be formed. Where the second material effectively encapsulates the first material, it is necessary to remove all of the second material which surrounds and encapsulates the first material in the region where the opening is to be formed.

Preferably the probe of the sensor (which is able to measure the parameters and/or analytes which the sensor is used to sense) is located adjacent to the opening formed by selective removal of the second material. This allows sensing of the environment in the region of the opening on the tube wall. For example, where the sensor is a glucose sensor, glucose is able to pass from the blood vessel or other cavity where the sensor is introduced through the opening and into the tube where its presence can be detected and measured by the probe.

The size of the opening in the outer wall will generally be between 1 and 400 mm², for example between 25 and 225 mm². The size of the opening must not be too small otherwise the blood or other substance into which the sensor is introduced will not be able to pass through the opening or will pass through in insufficient quantities for an accurate measurement to be made. The opening must also be large enough to allow positioning of the probe such that it is adjacent to the opening, even if it moves slightly when the sensor is introduced into the body.

In one embodiment of the invention, only one opening is generated in the tube wall, i.e. only one region of the second material is selectively removed. Preferably the opening extends only a portion of the way around the circumference of the tube: it is preferred to retain some continuity of the second material along the entire length of the tube, and is hence preferred that the opening generated by removal of the second material does not extend fully around the circumference of the tube. For example, it may be preferred that the opening extends around up to a maximum 75%, more preferably up to 50%, of the circumference of the tube.

In another embodiment of the invention, a plurality of openings can be generated in the tube wall, i.e. more than one region of the second material can be selectively removed. This embodiment allows for probes to be located at a number of points along the length of the tube, and for multiple measurements to be taken. Thus, it is possible for a number of probes to be located within the tube, each tube being adjacent to a different opening within the tube wall. Alternatively, a single probe could be located within the tube and be provided with means for moving it from one opening to another opening, hence allowing measurements to be taken at a number of points along the length of the tube.

The second material can be selectively removed by a number of different processes. The most suitable process will depend upon the nature of both the first and second materials, and much be chosen in order that the second material is removed in the region of interest while retaining the first material in that region. Suitable methods include removal of the second material by laser ablation, solvent, heat or abrasion. The most accurate technique is laser ablation, and this is preferred. Where the second material is removed by solvent, a solvent is chosen in which the second material is at least partially soluble. By exposing the region to be removed to the solvent, the second material present in that region effectively dissolves in the solvent, with the first material remaining.

The diameter of the tubes used in the invention varies according to the intended use of the resulting sensor. For example, sensors for introduction into a blood vessel generally have a relatively small diameter, reducing the disruption of the blood flow around the sensor. Accordingly, the tubes for use in the sensors must be engineered to be as small as possible. On the other hand, sensors for introduction into tissue or into a body cavity can generally be much larger, but again need to be biocompatible with the region into which they are introduced.

Suitable tubes of the invention generally have a diameter of up to 1 mm, for example from 0.01 to 1 mm, more preferably from 0.1 to 0.8 mm, more preferably from 0.2 or 0.25 mm to 0.7 or 0.75 mm, most preferably from 0.25 to 0.5 mm.

The sensor of the invention comprises a porous tube as described above, wherein the tube houses a probe comprising at least one analyte sensor. The probe is preferably located adjacent to the region of the tube where the second material has been selectively removed.

The sensors of the invention can be used to sense and measure a wide range of parameters and analytes. For example, they may be used to detect the presence and/or measure the concentration of a number of substances present in the body such as glucose, fructose, potassium, urea, creatinin and thiamine. Receptors for a number of analytes which could be incorporated into such a sensor are known in the art. For example, crown ethers may be used to detect potassium and various enzymes are also useful. In the case of saccharides, particularly glucose, a useful receptor is a boronic acid compound having a fluorophore. The boronic acid species provides the ability to complex with glucose and the fluorescence emission pattern of the molecule is altered in the presence of glucose, which allows optical detection.

The sensors of the invention may also be used to measure a number of other parameters which it may be desirable to monitor, for example oxygen (pO₂), carbon dioxide (pCO₂), hydrogen ion concentration (i.e. pH) and/or temperature.

Most preferably the sensors of the invention are used to measure glucose concentration. The present invention will be described further with reference to a particular type of invasive glucose sensor, but it should be understood that the invention is not limited to such sensors. Monitoring of patient glucose levels is particularly useful in intensive care units. It has been found that intensive care patients tend to have very high glucose levels. Mortality rates can be significantly reduced merely by maintaining normal glucose levels by administration of insulin. If, however, the patient is administered too much insulin then there is a risk of hypoglycaemia. Intermittent monitoring of glucose is not sufficient to prevent hypoglycaemia since the time from sampling to ascertaining a result is generally too long to accurately determine the current status of a patient, and their response to any administered insulin. Further, in vitro intermittent monitoring significantly increases the workload for the nursing staff due to the frequency of testing required. Invasive devices which provide continuous glucose monitoring are therefore particularly useful in the intensive care environment.

In addition to glucose, the sensors of the invention can also be used to measure a further parameter or analytes. For example, suitable sensors also measure one or more of temperature, pO₂, pCO₂ and pH, in addition to glucose levels. More preferably the sensors are used to measure both glucose levels and temperature.

Preferably the sensors of the invention comprise a porous tube as described above, wherein the tube houses a probe comprising an analyte sensor and a fibre-optic cable, the fibre-optic cable transporting the signal received from the analyte sensor to a receiving device (e.g. a transducer) which converts the signal into an electronic digital signal which can then be displayed and interpreted. The analyte sensor is preferably a glucose sensor. More preferably the probe also comprises a temperature sensor. In some embodiments it may also be desirable to include a pressure sensor within the probe. However, this generally increases the diameter of the tube which can be less preferred.

The sensors of the invention can be designed for a number of different applications and for introduction into a number of cavities, vessels and tissues. Preferably the sensors are designed to be introduced into blood vessels or into tissue, more preferably into blood vessels.

### EMBODIMENT 1

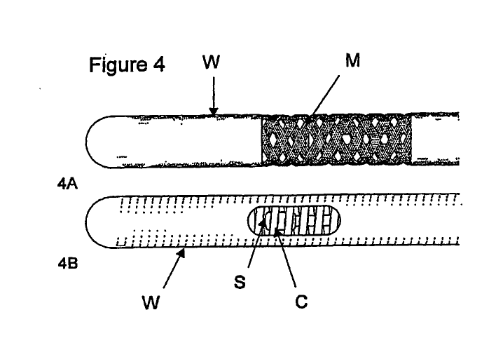

A first particular embodiment of the invention is depicted in FIG. 2A. This figure shows the mesh M of a first material which is coated with an outer wall W of a second material. Two regions R in which the outer wall of the second material has been removed are provided. On insertion of the device of FIG. 2A into a patient, material to be analysed (e.g. blood) can diffuse into the tubing at both of the open regions R.

A sensor S is located close to one of the open regions R such that material diffusing into the tube easily reaches the sensor. The open regions R extend only a part of the way around the circumference of the tube such that the open regions appear as “windows” in the outer wall.

A variation of this embodiment is depicted in FIG. 2B. This embodiment is the same as that described above with the exception that a single open region R is provided, and that the open region extends around the entire circumference of the tube wall.

### EMBODIMENT 2

A further embodiment of the invention is depicted in FIG. 3. In this embodiment the first material is in the form of a coil C which is coated with an outer coating or wall W of the second material. A single open region R in the outer wall is provided and the sensor probe S is located in the vicinity of this opening.

### EMBODIMENT 3

FIG. 4 depicts an alternative embodiment of the invention having a less porous mesh or coil. FIG. 4A shows a tube having a densely packed mesh M made of the first material and coated with an outer wall W of the second material. An open region in the outer wall W can be seen which extends around the entire circumference of the tubing. In this case, the mesh M shows a high density of filament crossovers. This embodiment therefore has an increased strength and a reduced porosity.

FIG. 4B depicts an embodiment in which the first material is in the form of a coil C which is coated with an outer wall W of the second material. A single open region is provided which extends only a part of the way around the circumference of the tubing. A sensor probe S is located in the vicinity of the open region. In this embodiment, the coil is densely packed, providing increased strength and reduced porosity in a similar manner to the embodiment depicted in 4A.

The invention has been described with reference to various specific embodiments and examples. However, it is to be understood that the invention is in no way limited to these specific embodiments and examples.

1. A process for preparing a tube for insertion into the human or animal body, said process comprising:
(a) providing a hollow tube comprising a first material and a second material, the first material being in the form of a coil or a tubular mesh, and the second material coating the first material in order to form a continuous substantially impermeable outer wall of the hollow tube;
(b) selectively removing a portion of said second material in order to generate at least one opening in a region of the outer wall, while retaining the first material in said region.

2. A process as claimed in claim 1 wherein the second material comprises a polymeric material.

3. A process as claimed in claim 2 wherein the second material comprises a synthetic polymeric material selected from polyamides, polyesters, polyurethanes, polyolefins, fluoropolymers and mixtures thereof.

4. A process as claimed in claim 1 wherein the second material is selectively removed by laser ablation.

5. A process as claimed in claim 1 wherein the first material comprises at least one metal selected from stainless steel, titanium and gold.

6. A process as claimed in claim 1 wherein the first material is in the form of a tubular mesh, said mesh being formed from a plurality of filaments.

7. A process as claimed in claim 6 wherein the filaments are arranged in order to define a mesh structure having multiple open areas of at least 0.3 cm² per cm² of the mesh structure.

8. A process as claimed in claim 6 wherein the first material comprises a plurality of helically wound filaments, at least a first said filament extending helically in the opposite sense to at least a second said filament.

9. A process as claimed in claim 1 wherein the first material forms a tubular mesh in which the mesh structure is a braid.

10. A process according to claim 1 wherein the first material is in the form of a coil, and wherein the size of the opening formed by selective removal of the second material can be varied by extending or compressing said coil.

11. A process according to claim 1 wherein the second material is selectively removed in the shape of an arc extending around at least a portion of the circumference of the tube.

12. A process as claimed in claim 11 wherein the arc formed by selective removal of the second material does not extend around the complete circumference of the tube.

13. A process as claimed in claim 1 wherein the second material is selectively removed in the shape of a strip or strips extending longitudinally along at least a portion of the tube.

14. A tube for insertion into the human or animal body which is preparable according to a process comprising:
(a) providing a hollow tube comprising a first material and a second material, the first material being in the form of a coil or a tubular mesh, and the second material coating the first material in order to form a continuous substantially impermeable outer wall of the hollow tube;
(b) selectively removing a portion of said second material in order to generate at least one opening in a region of the outer wall, while retaining the first material in said region.

15. A tube for insertion into the human or animal body, said tube comprising a first material in the form of a coil or a tubular mesh, and a second material coating the first material in order to form a continuous substantially impermeable outer wall of the hollow tube, wherein at least a portion of the second material has been selectively removed in order to generate at least one opening in a region of the outer wall, while retaining the first material in said region.

16. A tube as claimed in claim 15 wherein the opening generated by selective removal of the second material extends only part of the way around the circumference of the tube.

17. A sensor device comprising a tube for insertion into the human or animal body, said tube comprising a first material in the form of a coil or a tubular mesh, and a second material coating the first material in order to form a continuous substantially impermeable outer wall of the hollow tube, wherein at least a portion of the second material has been selectively removed in order to generate at least one opening in a region of the outer wall, while retaining the first material in said region, wherein said tube houses a probe comprising at least one analyte sensor.

18. A sensor device as claimed in claim 17 wherein the probe can be positioned adjacent to the opening generated in the outer wall due to selective removal of the second material.

19. A sensor device as claimed in claim 17 wherein the at least one analyte sensor is a sensor for measuring glucose concentration.

20. A sensor device as claimed in claim 19 which comprises a first sensor for measuring glucose and a second sensor for measuring temperature.
